(19) United States
(12) Patent Application Publication
(10) Pub. No.: US 2025/0032315 A1
(43) Pub. Date: Jan. 30, 2025

(54) VITREOUS ASPIRATION AND DRUG DELIVERY DEVICE

(71) Applicant: TWENTY TWENTY THERAPEUTICS LLC, South San Francisco, CA (US)

(72) Inventor: Alexandre R. Tumlinson, South San Francisco, CA (US)

(21) Appl. No.: 18/780,293

(22) Filed: Jul. 22, 2024

Related U.S. Application Data
(60) Provisional application No. 63/528,640, filed on Jul. 24, 2023.

(51) Int. Cl. A61F 9/007; A61F 9/00

(52) U.S. Cl. CPC A61F 9/00736; A61F 9/0008

(57) ABSTRACT

A vitrectomy device includes a carrier having a pocket. A cover is connected to the carrier with at least one of the carrier and the cover has an axle defining an axis. A trigger is pivotably connected to the axle and having a passage. A hollow needle is provided for insertion into the vitreous humor of an eye and extends within the passage. A vacuum sample assembly is received in the pocket and has a chamber under vacuum. The trigger is pivotable about the axle from a first position in which the chamber is not fluidly connected to the needle to a second position in which the needle is bent into fluid communication with the chamber for automatically drawing a vitreous sample through the needle into the chamber under vacuum pressure. Other aspects are also described.

### RELATED APPLICATIONS

This nonprovisional patent application claims the benefit of the earlier filing date of U.S. provisional application No. 63/528,640 filed 24 Jul. 2023.

### TECHNICAL FIELD

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites, and nutrients. It is produced largely by cells of the ciliary body. Changes in vitreous structure that occur with aging are important in the pathogenesis of many vitreoretinal diseases.

Intraocular pressure (IOP) quantifies the pressure of the vitreous humor inside the eye. Many individuals suffer from disorders, such as glaucoma, which are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection. Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one week. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to a depth of approximately 6-11 mm, injecting the drug, withdrawing the needle, and allowing the conjunctiva to cover the injection site. Post injection care typically includes a basic verification of functional vision such as requesting the patient to count the number of fingers shown by the doctor. This functional test verifies that acute IOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauges may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase IOP by as much as 25 mmHg, which is substantially greater than threshold levels that are considered potentially harmful. Evidence shows that while such IOP increases are transient, they are in fact associated with an iatrogenic glaucoma resulting in measurable loss of nerve fiber layer and visual function over a course of only several treatments in patients with ‘normal’ resting IOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required.

### SUMMARY

In one example, a vitrectomy device includes a carrier having a pocket. A cover is connected to the carrier with at least one of the carrier and the cover has an axle defining an axis. A trigger is pivotably connected to the axle and having a passage. A hollow needle is provided for insertion into the vitreous humor of an eye and extends within the passage. A vacuum sample assembly is received in the pocket and has a chamber under vacuum. The trigger is pivotable about the axle from a first position in which the chamber is not fluidly connected to the needle to a second position in which the needle is bent into fluid communication with the chamber for automatically drawing a vitreous sample through the needle into the chamber under vacuum pressure.

In another example, a vitrectomy device includes clip having a pocket and a vacuum sample assembly received in the pocket with a chamber under vacuum. A carrier has an axle for pivotably receiving the clip. A needle mount is pivotably mounted on the axle and includes a passage. A hollow needle is provided for insertion into the vitreous humor of an eye and extends within the passage. A cam system is configured to convert pivotal movement of clip into pivotal movement of the needle mount. The needle has a first condition fluidly isolated from the chamber when the clip has a first position relative to the needle mount and a second condition in which the needle is fluidly connected with the chamber in response to pivotal movement of the clip in a first direction for drawing a vitreous sample through the needle into the chamber under vacuum pressure.

In another example, a vitrectomy device for an eye includes a carrier and a hollow needle for insertion into the vitreous humor of the eye. The needle extends from an upstream end to a downstream end and through the carrier. A vitreous sample collection assembly is coupled to the carrier at a first location. A drug container is coupled to the carrier at a second location downstream of the vitreous sample collection assembly relative to the extension of the needle. The upstream end of the needle has a first condition passing through the drug container and spaced from the sample collection assembly, a second condition extending into the sample collection assembly for removing a vitreous sample from the eye, and a third condition spaced from the sample collection assembly and positioned within the drug container for delivering a therapeutic agent in the drug container to the eye.

Other objects and advantages and a fuller understanding of the invention will be had from the following detailed description and the accompanying drawings.

### BRIEF DESCRIPTION OF THE DRAWINGS

FIG. 1 is a schematic illustration of an example combined biological sampling and injection device in accordance with the present invention.

FIG. 2 is an exploded view of a syringe of the device.

FIG. 3A is a front view of a trigger of the device.

FIG. 3B is a rear view of the trigger.

FIG. 4 is a front view of a cover of the device.

FIG. 5A is a front view of a carrier of the device.

FIG. 5B is a rear view of the carrier.

FIG. 6A is a front view of a relief cap of the device.

FIG. 6B is a rear view of the relief cap.

FIG. 7A is a schematic illustration of a subassembly of the device.

FIG. 7B is a section view of the subassembly of FIG. 7A taken along line 7B-7B.

FIG. 7C is a section view of the subassembly of FIG. 7B taken along line 7C-7C.

FIGS. 8A-8C are schematic illustrations depicting steps of operating the device.

FIG. 9 is a schematic illustration of another example combined biological sampling and injection device in accordance with the present invention.

FIG. 10 is a schematic illustration of a portion of a cam system of the device.

FIG. 11 is an exploded view of FIG. 10.

FIG. 12 is a schematic illustration of another portion of the cam system.

FIG. 13A is a front view of a needle mount of a portion of FIG. 12.

FIG. 13B is a section view of the needle mount of FIG. 13A.

FIG. 14 is a front view of a slider of the portion of FIG. 12.

FIG. 15A is a front view of the assembled cam system.

FIG. 15B is an enlarged view of the assembly cam system.

FIG. 16 is a schematic illustration of a relief cam of the device of FIG. 9.

FIG. 17 is a schematic illustration of the device of FIG. 9 in a first condition.

FIG. 18 is a schematic illustration of the device of FIG. 9 in a second condition.

FIG. 19 is a schematic illustration of the device of FIG. 9 in a third condition.

FIG. 20 is a schematic illustration of another example relief cap.

FIGS. 21A-21C are a schematic illustration of operation of the device of the present invention.

FIGS. 22A-22C are a schematic illustration of alternative operation of the device of the present invention.

### DETAILED DESCRIPTION

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

The present invention operates on the premise that by positioning the sampling structure relative to the drug delivery structure in a particular manner, vitreous sampling and drug delivery can be readily performed while allowing for rapid removal of the sampling structure once the procedure is complete. In one instance, the vitreous sampling structure is positioned upstream or proximal to the drug containing structure and in an exposed location on the device. That said, the vitreous sample can be readily removed from the device without requiring manipulation of the drug container, disassembly of the device or otherwise additional steps to access and remove the sampling structure.

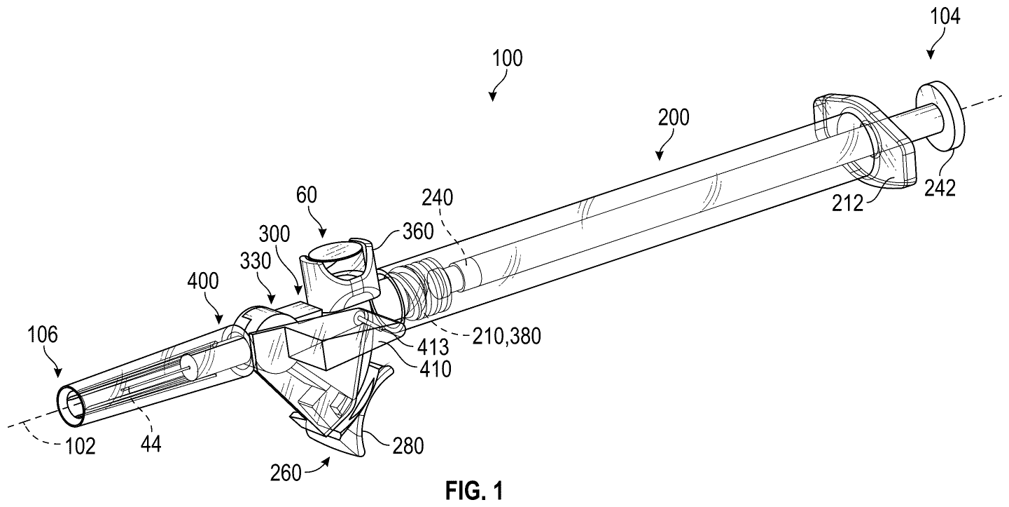

FIG. 1 illustrates an example combined biological sampling and injection assembly or device 100 in accordance with the present invention. The device 100 extends along a centerline 102 from a first or proximal end 104 to a second or distal end 106. The device 100 is operable by a user, e.g., a physician, to obtain a sample of vitreous humor from an eye of a patient. The device 100 includes, among other things, a syringe 200, a trigger 260, a cover 300, a carrier 330, and a relief cap 400. The components are oriented and configured to help sequentially acquire a vitreous sample from an eye and deliver a therapeutic agent or drug to the injection site, both via the same needle. In one example, the trigger 260 is used to bend the needle 80 to different positions within the device 100 in order to accomplish the respective steps.

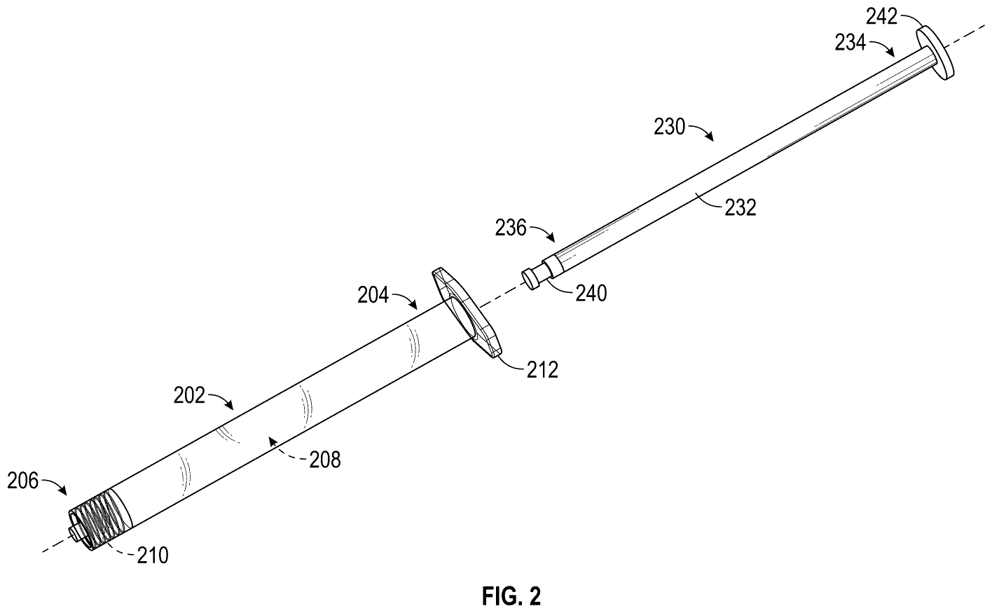

Referring to FIG. 2, the syringe 200 includes a tube 202 extending from a first end 204 to a second end 206. A passage 208 extends longitudinally the entire length of the tube 202. A connection 210, e.g., a luer-type connection, is provided at the second end 206. A handle 212 is provided at the first end 204. The tube 202 can be formed from a translucent material and includes gradations along its length.

The syringe 200 further includes a plunger assembly 230 having an elongated shaft 232 extending from a first end 234 to a second end 236. A stopper or plunger 240 is provided at the second end 236. A handle 242 is provided at the first end 234. The shaft 232 is configured to be slidably received within the passage 208 of the tube 202 with the plunger 240 providing a friction fit with the interior of the tube. A drug capsule, such as a therapeutic agent, (not shown) is provided in the passage 208 of the tube 202 distal/downstream of the plunger 240.

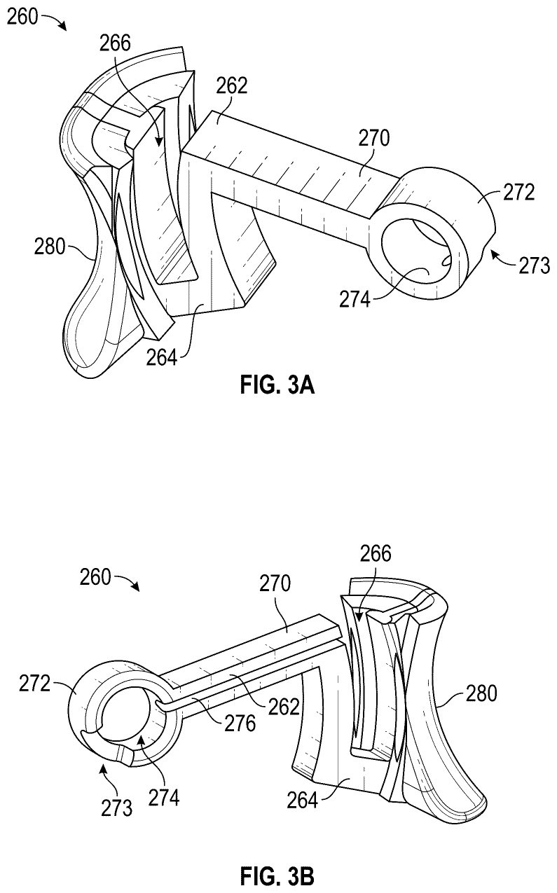

Turning to FIGS. 3A-3B, the trigger 260 includes an arm 262 having a first portion 264 and a second portion 270. As shown, the first portion 264 is generally U-shaped and the second portion 270 extends longitudinally away from the first portion 264. The first portion 264 defines a channel 266. A round, e.g., circular, projection 272 is formed at the end of the second portion 270 opposite the first portion 264. An opening 274 extends through the projection 272. A button 280 is formed on an end of the first portion 264 opposite the second portion 270. A passage 276 extends through the second portion 270 from the channel 266 to the opening 274.

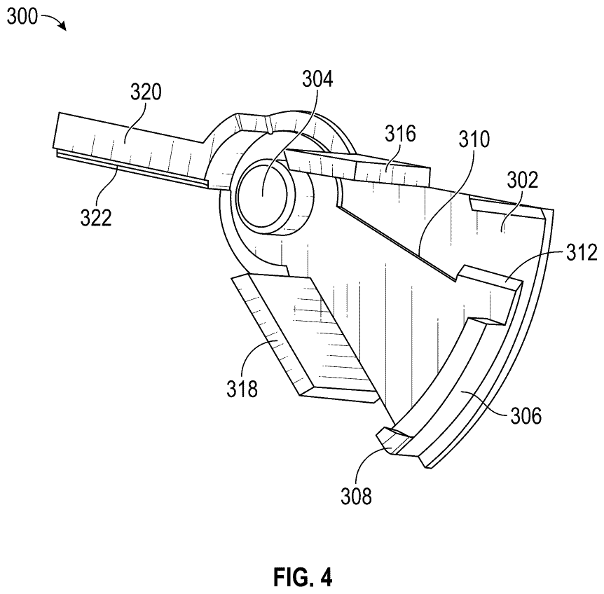

A cover 300 is shown in FIG. 4. The cover 300 includes a generally wedge-shaped base 302. An axle 304 extends from the base 302. A guide member 306 extends from the base 302 and circumferentially relative to the axle 304. The guide member 306 terminates at a tab 308. A shelf 310 on the base 302 extends generally radially from the axle 304. A ramp 312 on the base 302 extends generally radially from the shelf 310. A pair of arms 316, 318 extends from opposite sides of the base 302 and generally radially from the axle 304. An arm 320 extends away from the base 302 on the opposite side of the axle 304 from the arms 316, 318. A notch 322 extends the length of the arm 320. The arms 316, 318, 320 are circumferentially spaced apart from one another about the axle 304.

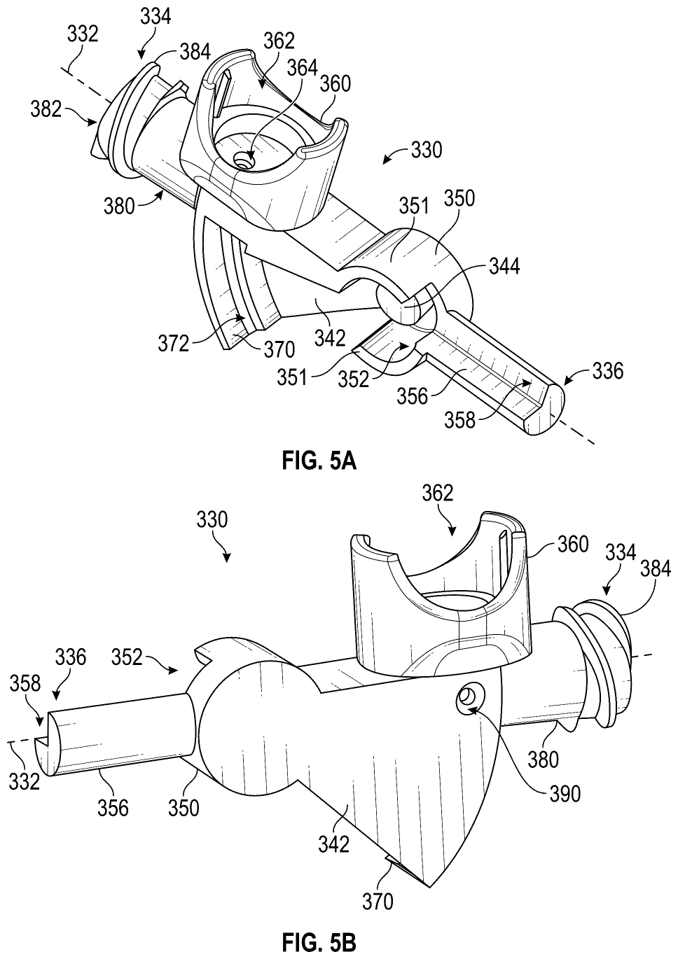

FIGS. 5A-5B illustrate the carrier 330. The carrier 330 extends along a centerline 332 from a first end 334 to a second end 336. The carrier 330 includes a generally wedge-shaped base 342. An axle 344 extends from the base 342. A first projection 350 extends from the base 342 and includes a pair of spaced-apart portions 351 extending circumferentially about the axle 344. To this end, the portions 351 define a recess 352 in which the axle 344 is provided. An arm 356 extends away from the base 342 on the opposite side of the axle 344 from a second projection 360. A notch 358 extends the length of the arm 356.

The second, cylindrical projection 360 extends upward (as shown) from the base 342. The second projection 360 is hollow and defines a pocket or recess 362. An opening 364 extends from the recess 362 to an opening 390 (FIG. 5B) extending laterally through the base 342.

A guide member 370 extends from the base 342 and circumferentially relative to the axle 344. As shown, the guide member 370 extends from the base 342 and circumferentially relative to the axle 344. A recess 372 is formed in the guide member 370 that also extends circumferentially relative to the axle 344. A luer connection 380 extends rearward (as shown) from the base 342 and defines a passage 382.

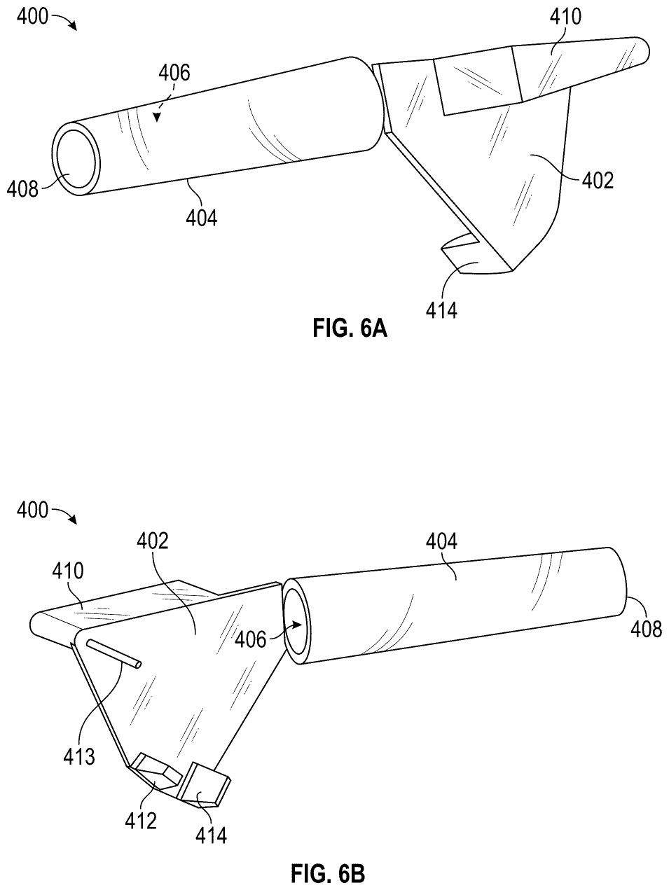

The relief cap 400 is illustrated in FIGS. 6A-6B. The relief cap 400 includes a generally triangular base 402 and an elongated projection 404 extending therefrom. The projection 404 is hollow and defines a passage 406 closed at a closed end 408. A hollow guide 410 extends laterally from the base 402. A pair of pins 412, 414 (or tabs) and a hollow relief needle extend laterally from the base 402 in a direction opposite to the guide 410.

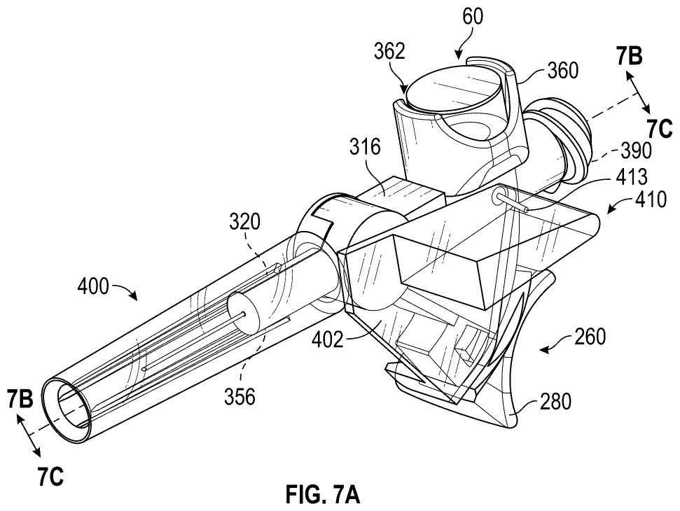

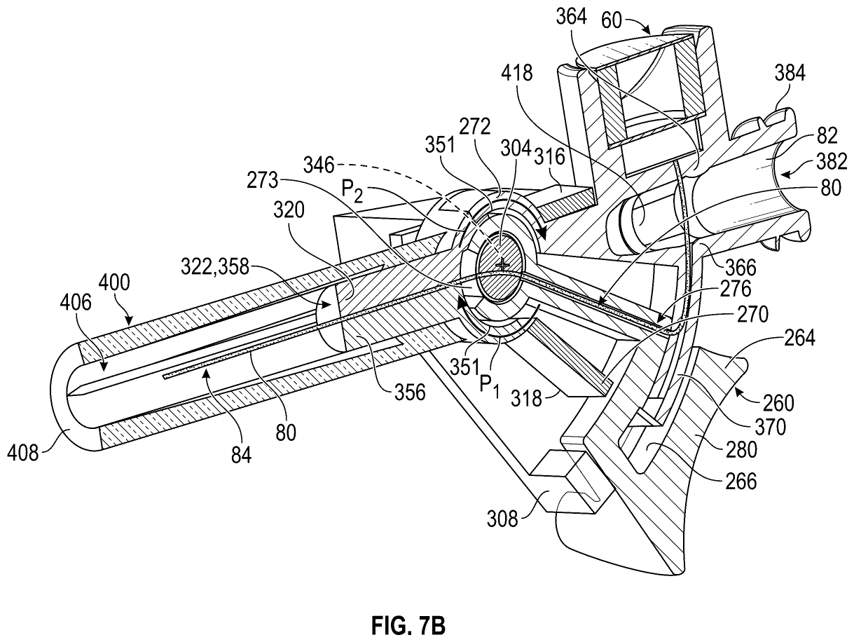

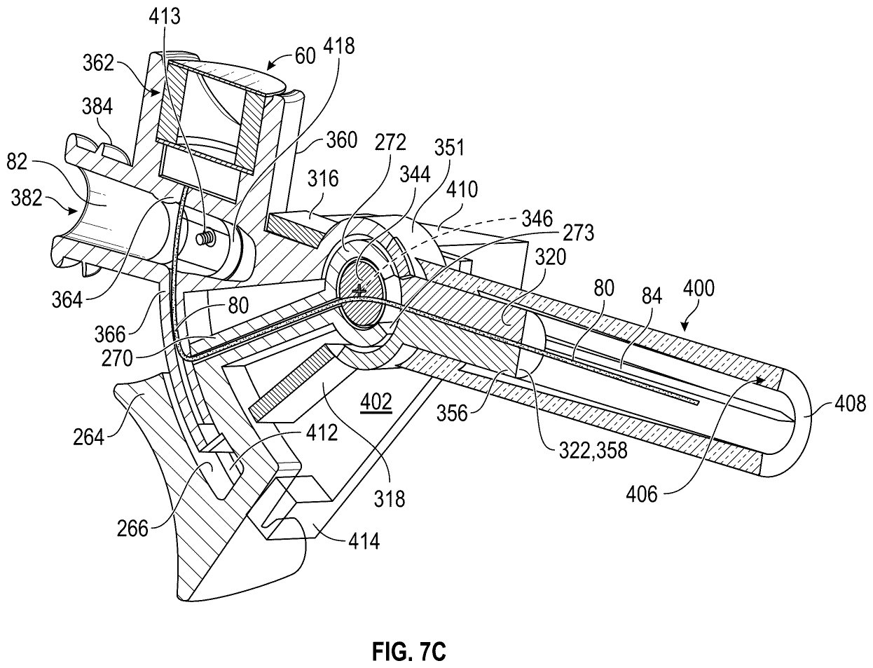

When the device 100 is assembled (FIGS. 7A-7C), the cover 300 and carrier 330 are aligned with and connect directly to one another. More specifically, the portions 351 of the first projection 350 on the carrier 330 are inserted in the gaps between the arms 316, 318, 320 of the cover 300. This aligns the respective axles 304, 344 with one another along a common pivot axis 346. At the same time, the arm 320 on the cover 300 and the arm 356 on the carrier 330 mate with another to enclose (but not obstruct) the notches 322, 358. The arms 316, 318 on the cover 300 overlay the base 342 of the carrier 330. The guide member 306 on the cover 300 extends into the recess 372 on the carrier 330.

The trigger 260 is positioned such that both axles 304, 344 are positioned within the opening 274 of the projection 272. This positions the projection 272 within the recess 352 of the first projection 350 of the carrier 330. The recess 273 in the projection 272 is adjacent and aligned with the notches 322, 358. The second portion 270 of the arm 262 extends between the bases 302, 342 and towards the guide members 306, 370. The first portion 264 extends between cover 300 and the carrier 330 to the exterior of the device 100. Due to the shape of the first portion 264, this positions the button 280 adjacent to the exterior of the guide members 306, 370. Consequently, the guide members 306, 370 extend into the channel 266 in the first portion 264 of the trigger 260.

Due to this construction, the trigger 260 is pivotable relative to the cover 300 and the carrier 330. More specifically, the button 280 can be urged in a first direction (counterclockwise as shown) towards the luer connection 380 to simultaneously cause the projection 272 to rotate about the axles 304, 344 while the second portion 270 pivots about the axles in the manner P₁. The trigger 260 is pivotable in the manner P₁ until reaching a first (retracted) position at the end of the base 342 abutting the carrier 330 adjacent the luer connection 380. Alternatively, or additionally, the trigger 260 reaches the first position when the guide members 306, 370 bottom out in the channel 266 of the trigger. In this instance, the degree to which the second portion 270 can pivot in the manner P₁ is therefore dictated by the initial circumferential gap between the end of the channel 266 and the leading surfaces of the guide members 306, 370.

The button 280 can also be urged in a second direction (clockwise as shown) away from the luer connection 380. This simultaneously causes the projection 272 to rotate about the axles 304, 344 while the second portion 270 pivots about the axles in the manner P₂. The trigger 260 is pivotable in the manner P₂ until the second portion 270 reaches a second (extended) position engaging the arm 318 on the cover 300. With this in mind, the device 100 can be configured such that the trigger 260 has a predetermined, pivotal range of motion between the first and second positions.

The relief cap 400 is positioned such that the arms 320, 356 extend into the projection 404. The base 402 extends over the exterior of the base 342 of the carrier 330. The pin 414 abuts the outer surface of the first portion 264 of the trigger 260. The tab 412 extends into the channel 266 in the trigger 260 between the first portion 264 and the guide member 370 on the carrier 330. In one example, the pins 412, 414 can have snap-in or friction fit engagements/connection with the first portion 264 and/or guide member 370 to help temporarily hold the relief cap 400 on the remainder of the device 100. The hollow guide 410 is placed in fluid communication with the lateral opening 390 through the hollow relief needle 413 extending through both the lateral opening 390 and the hollow guide (see FIGS. 1 and 7A).

A vacuum sample assembly 60 is provided in the recess 362 of the carrier 330. The vacuum sample assembly 60 includes a clear/transparent container 62 defining a sample chamber 64. The sample chamber 64 is evacuated of air and is therefore at vacuum pressure. The wall of the sample chamber 64 can have surface indents or a roughness that cause a lensing or scattering effect when the chamber is empty, but effectively disappear when the chamber is filled with a clear fluid with an index of refraction more closely matched to the clear material of the vacuum chamber. A compliant seal includes a cover or septum 66 for sealing the sample chamber 64. The septum 66 provides for long term maintenance of the vacuum in the sample chamber 64 during storage.

A flexible needle 80 extends from a first or proximal end 82 positioned within the opening 366 adjacent the septum 66 to a second or distal end 84 positioned longitudinally beyond the arms 320, 356 and within the passage 406 of the relief cap 400. More specifically, the needle 80 extends from the opening 364, into the passage 382, through an elastomeric member 418 or seal positioned within the passage 382, through the other opening 366 generally aligned with the opening 364, through a gap between the guide members 306, 370, through the passage 276 in the first portion 264 of the trigger 260, between the axles 304, 344, through the recess 273 in the projection 272, and through the notches 322, 358 in the cooperating arms 320, 356 into the passage 406.

The flexible configuration of the needle 80 allows for this tortured contour through the device 100 without kinking or damaging the needle. That said, a central lumen extends the entire length of the needle 80. The second end 84 of the needle 80 has a sharpened tip configured for insertion into or through at least one of the sclera, the pars plana, the anterior segment or the vitreous body of an eye.

The luer connections 210, 380 on the respective syringe 200 and carrier 330 are mated with one another to secure the syringe 200 to the carrier. This places the passage 208 of the tube 202 in fluid communication with the passage 382 in the carrier.

In operation, in advance of the vitrectomy the user can rely on the transparency of the tube 202 to ensure the drug is provided therein. The syringe 200 is pre-filled with the drug and, thus, the elastomeric member 418 acts as a drug container as it is filled with medication. The lateral opening 390 can act to direct air bubbles and overflow drug through the relief needle 413 to the hollow guide 410. The user can remove the relief cap 400 to expose the distal end 84 of the needle 80. At the same time, this removes the pins 412, 414 from either side of the first portion 264 of the trigger 260. Simultaneously, the relief needle 413 is withdrawn from the lateral opening 390. Consequently, the device 100 in this state is considered to be “ready” or “armed” and therefore operable. It will be appreciated that prior to removing the relief cap 400 the pins 412, 414 prevent any movement of the trigger 260 such that device is considered to be “disarmed” and therefore inoperable.

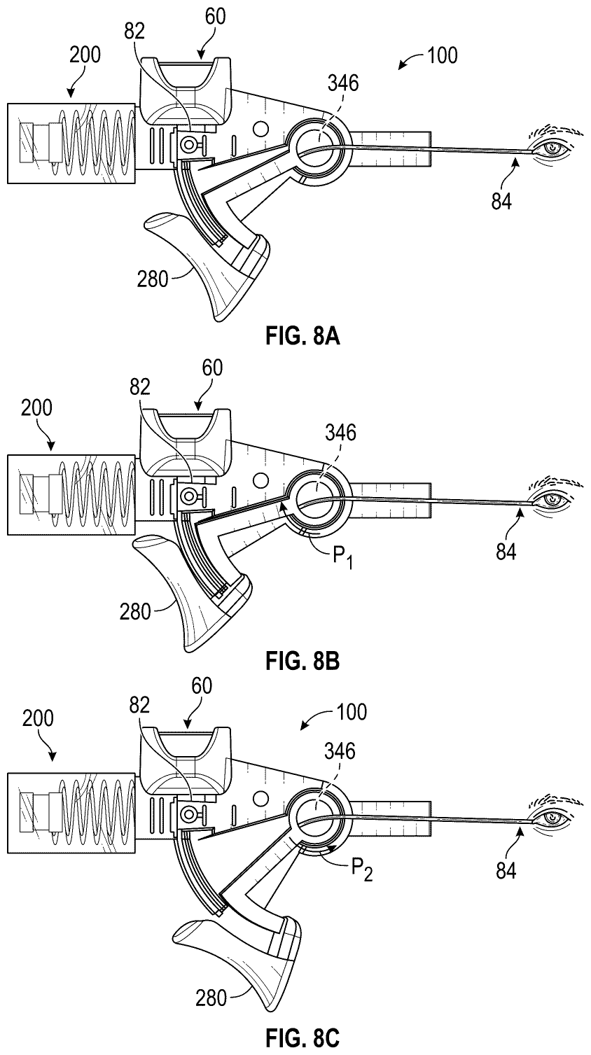

Turning to FIGS. 8A-8C, the distal end 84 of the needle 80 is advanced into the vitreous humor of the eye (not shown). The user then actuates the trigger 260 by pivoting it in the manner P₁. When this occurs, the pivoting trigger 260 elastically deforms the portion of the needle 80 between the axles 304, 344 and the first end 84. More specifically, this portion of the needle 80 is bent in a first direction (counterclockwise as shown) about a point at or substantially at the pivot axis 346.

It will be appreciated that the portions of the needle 80 on either side of the axles 304, 344 are confined/enclosed and, thus, substantially prevented from bending during this actuation. In particular, the portion of the needle 80 distal to the axles 304, 344 is held within the notches 322, 358 while the portion of the needle proximal to the axles is held within the passage 276 of the trigger 260. The notches 322, 358 and passage 276 may allow for some movement of the needle 80 therein but in any case, elastic deformation of the needle 80 during actuation is substantially limited to the portion adjacent/aligned with the pivot axis 346 to help maintain the integrity of the needle.

With this in mind, the first end 82 of the needle 80 passes through the seal 66 and into the sample chamber 64 as or just prior to the trigger 260 reaching the first position. This places the sample chamber 64 in fluid communication with the lumen of the needle 80, which thereby allows the vacuum within the chamber to automatically disrupt the integrity of the vitreous humor. More specifically, the vacuum breaks apart and liquefies the vitreous humor sufficiently to draw portions of the vitreous humor into the lumen of the needle 80 and ultimately into the sample chamber 64.

Once the sample chamber 64 is filled, the user actuates the trigger 260 in the manner P₂ until it reaches the second position extended away from the remainder of the device 100. When this occurs, the needle 80 is bent in a second direction (counterclockwise as shown) at or substantially at the pivot axis 346. Consequently, the first end 82 of the needle 80 is moved out of the sample chamber 64, back through the seal 66, and into the passage 382 of the carrier 330. The seal 66 is self-closing and, thus, the acquired vitreous sample is retained within the sample chamber 64 after the needle 80 is removed therefrom.

It will be appreciated that the recess 273 in the projection 272 has a radial arc wide enough that the projection does not abut or bend the portion of the needle 80 distal to the axles 304, 344 (rightward as shown in FIG. 8C) during actuation of the trigger 260 in either manner P₁, P₂. In other words, the projection 272 does not engage the portion of the needle 80 leading into the notches 322, 358 during actuation of the trigger 260 in either manner P₁, P₂, thereby limiting the extent to which the needle is bent during actuation.

That said, placing the first end 82 of the needle 80 within the passage 382 places it in fluid communication with the passage 208 of the syringe 200 and, by extension, in fluid communication with the drug (not shown) therein. The user can then use the handle 242 to advance the plunger 240 via sliding movement towards the carrier 330 in the direction D and relative to the tube 202. The advancing plunger 240 pressurizes and pushes the drug into the passage 382 in the carrier 330, into the first end 82 of the needle 80, through the entire length of the needle, and ultimately out of the second end 84 of the needle to be fully injected into the eye where the vitreous humor sample was removed. As noted, the tube 202 can be formed from a transparent or semi-transparent material to enable the user to confirm that the drug has been delivered by looking through the lateral openings 32 in the body 20 and/or the lateral passages in the cap. Additionally, the vacuum sample chamber 60 can be removed from the recess 352, following pivoting the trigger to the second position, following drug delivery or following removal of the device 100 from the eye. The remainder of the device 100 can then be discarded.

Regardless, it will be appreciated that because the vacuum sample chamber 60 is positioned upstream of the drug-filled elastomeric member 418, the former can readily be removed from the device 100 without any need to adjust, move, or otherwise disturb the elastomeric member. In other words, the vacuum sample chamber 60 is not positioned in the device 100 in a hard to reach or cumbersome position. This advantageously drastically reduces the time and effort needed to remove the acquired vitreous sample from the device 100 prior to discarding it.

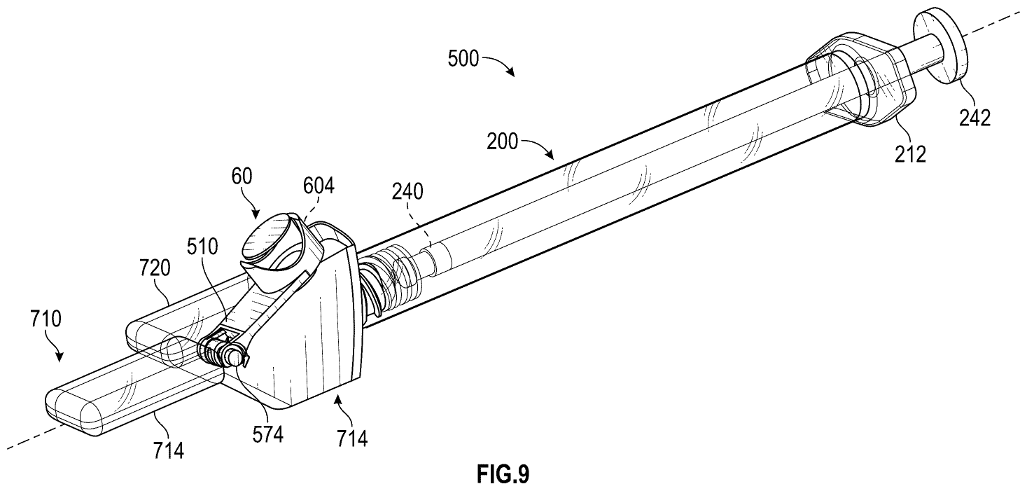

Another example, device 500 is illustrated in FIG. 9. Features in FIG. 9 that are identical to features in FIGS. 1-8C are given the same reference number. The device 500 includes a spring biased cam system 510 for helping to control both vitreous sample removal and subsequent drug delivery to the removal site.

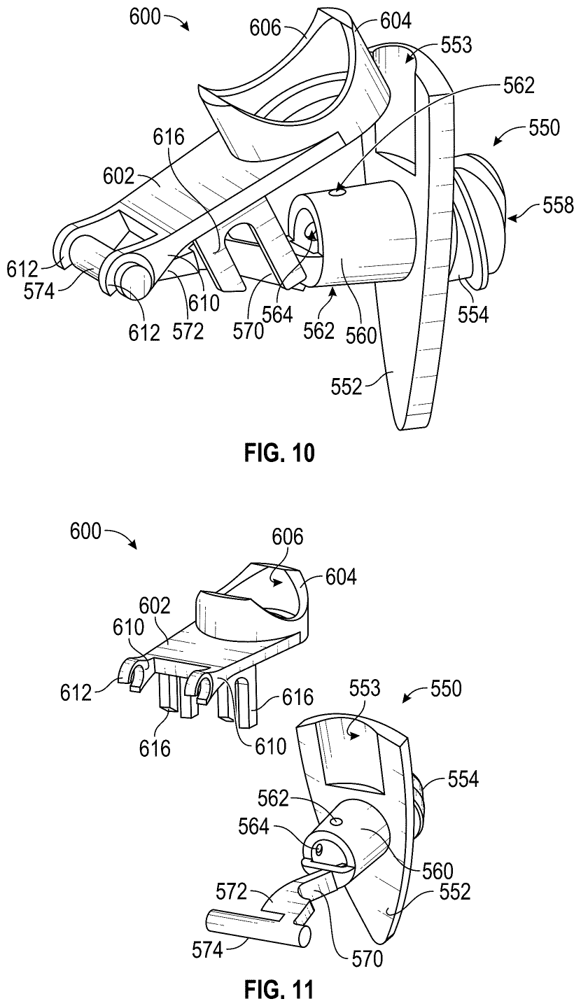

A portion of the cam system 510 is illustrated in FIGS. 10-11. To this end, the cam system 510 includes a carrier 550 having a base 552. In one example, the base 552 is wedge-shaped and includes a recess or pocket 553. A luer connection 554 extends from the base 552 and defines a passage 558. A hollow projection 560 extends from the base 552 and away from the luer connection 554. A pair of openings 562 are aligned vertically with one another and extend through the projection 560. A lateral opening 564 also extends through the projection 560 and between the pair of openings 562. It will be appreciated that the elastomeric member 418 occludes both of the openings 562 when positioned within the luer connection 554. The lateral opening 564 is configured to receive the relief needle 413 and is also occluded by the elastomeric member 418. An arm 570 extends from the projection 560 and includes an angled or ramped portion 572. An axle 574 is secured to the angled portion 572 and extends generally perpendicular thereto.

The cam system 510 further includes a trigger or clip 600 having a generally planar base 602. A projection 604 extends from the base 602 and defines a pocket 606 for receiving the vacuum sample chamber 80. A pair of arms 610 extends from the base 602 and generally away from the projection 604. Each arm 610 terminates at a hook or clip 612. The clips 612 are generally C-shaped and configured to grasp opposite ends of the axle 574 (see FIG. 10). A pair of guide members 616 extends from the base 602 (downward as shown). The guide members 616 can be generally U-shaped and can extend transverse, e.g., perpendicular, to the clips 612.

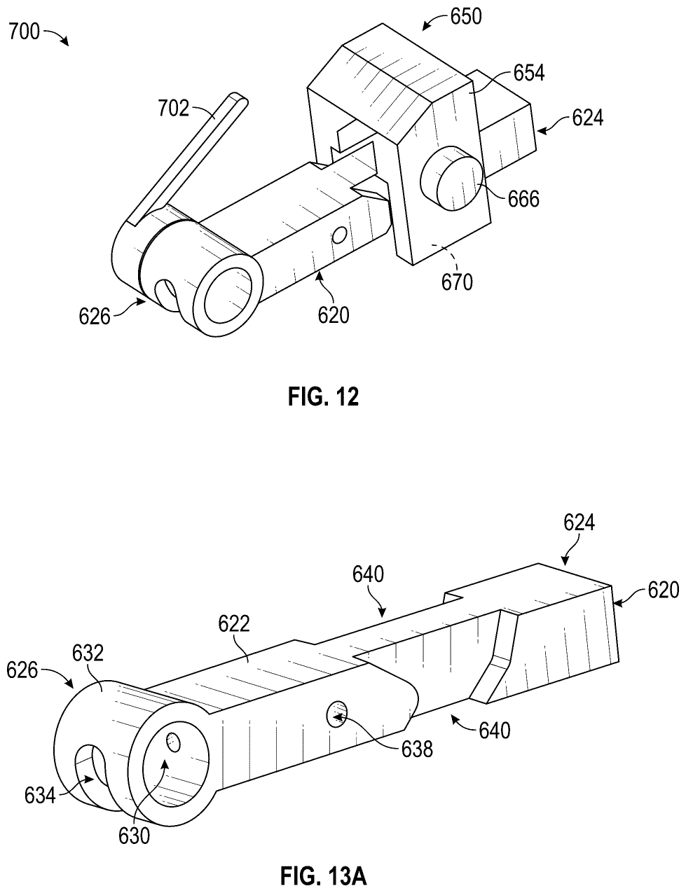

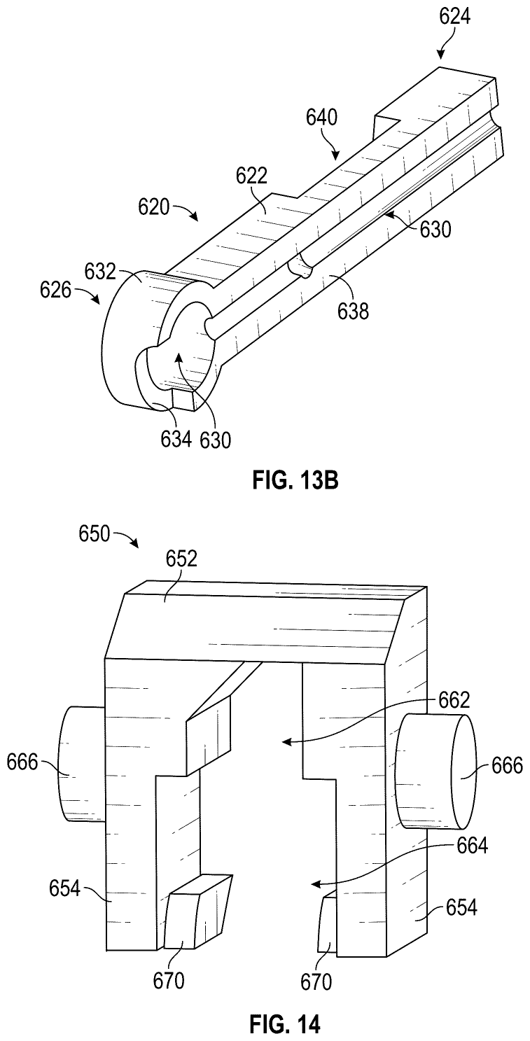

Additional components of the cam system 510 are illustrated in FIGS. 12-14. To this end, the cam system 510 further includes a needle mount 620, a slider 650, and a biasing member, such as a spring 700 having a pair of arms 702, 704. Turning to FIGS. 13A-13B, the needle mount 620 includes a generally rectangular base 622 that extends from a first end 624 to a second end 626. A passage 630 extends the length of the base 622. A cylindrical tube 632 is provided on the second end 626 and extends generally perpendicular to the base 622. The passage 630 extends to the interior of the tube 632. A notch or opening 634 extends through the tube 632 and is longitudinally aligned with the passage 630. A lateral passage 638 extends through the base 622 and intersects the passage 630. Guide members 640, e.g., recesses, are formed on opposing sides of the base 622 adjacent the first end 624.

The slider 650 shown in FIG. 14 is generally U-shaped and includes a base 652 and a pair of legs 654 extending therefrom. The legs 654 are spaced apart to define a receiving space therebetween having a first portion 662 and a second portion 664. First projections 666 extend from the legs 654 and away from one another. The first projections 666 are aligned with one another. Second projections 670 extend from the legs 654 and towards one another into the second portion 664 of the receiving space. As shown, the first projections 666 are cylindrical and the second projections 670 have a generally parallelogram shape. Other shapes for the first and second projections 666, 670 are contemplated.

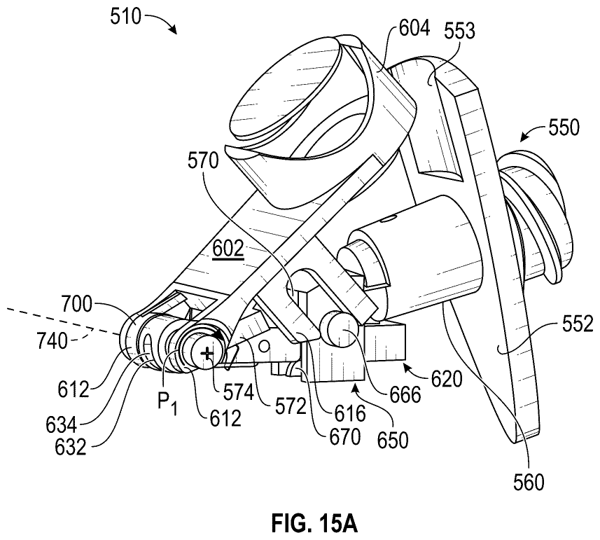

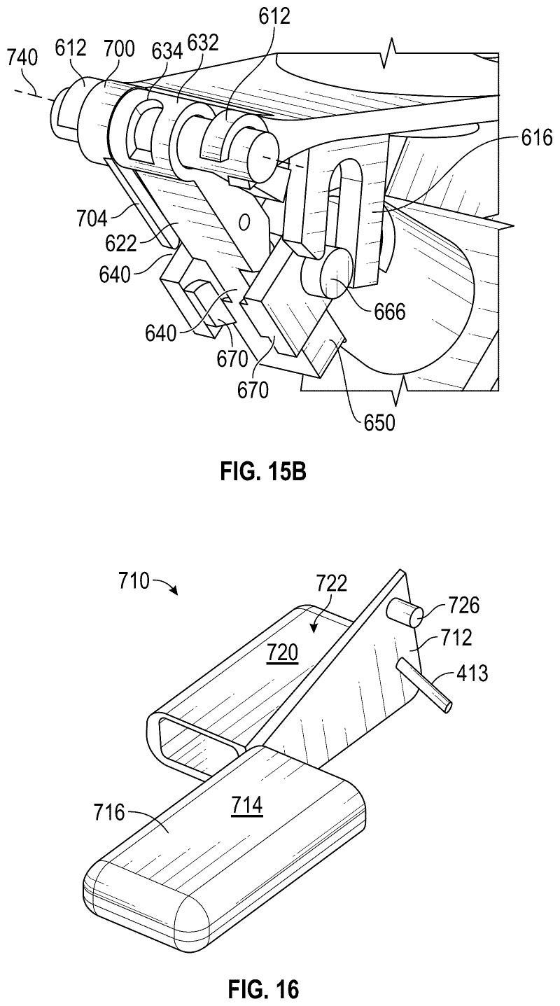

The fully assembled cam system 510 is shown in FIGS. 15A-15B. In this configuration, the axle 574 extends through the spring 700, the tube 632, and the clips 612. The arm 570 is received in the first portion 662 of the receiving space of the slider 650 such that the slider rests atop and is slidable relative to the arm. The needle mount 620 extends beneath (as shown) the arm 570 and within the second portion 664 of the receiving space of the slider 650 such that the legs 654 are generally aligned with the guide members 640 on the needle mount. In the position/condition shown, the second projections 670 are offset from the guide members 640 such that the needle mount 620 rests on the second projections. Furthermore, the guide members 616 on the clip 600 at least partially receive the respective first projections 666 on the slider 650.

The cam system 510 is received in the cover 520 such that the base 602 of the clip 600 extends across the top of the cover while the axle 574 is rotatably mounted to the cover.

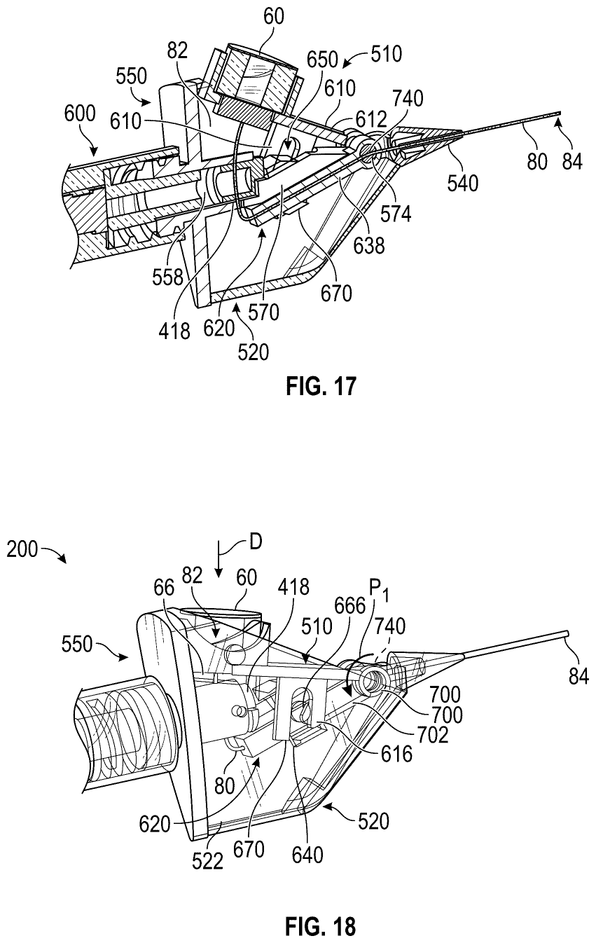

Turning to FIG. 17, the proximal end 82 of the needle 80 is positioned within the seal 66 on the vacuum sample chamber 60. The needle 80 then extends downstream, through the aligned openings 562 and the elastomeric member 418 in the projection 560, through the passage 630, the tube 632, and the opening 634 in the needle mount 620, and out through a guide member in the cover 520. The needle 80 is bent along its length to accommodate this tortuous path. With this in mind, the needle 80 has a radius of curvature aligned with or approximating the axis 740 of the axle 574.

A relief cap 710 is shown in FIG. 16 and includes a base 712. A pair of receptacles 714, 720 extend from the base 712, with each defining a respective chamber 716, 722. A projection 726 extends from the base 712. The relief needle 413 extends laterally from the base 712 for directing overflow drug from the elastomeric member 418 to the chamber 722.

The cam system 510 is designed such that actuation of the clip 600 by the user controls/facilitates both vitreous sample acquisition and drug delivery. As noted, and turning to FIGS. 17-19, the second projections 670 on the slider 650 are initially offset from the guide members 640 in the needle mount 620, thereby preventing the needle mount from pivoting about the axis 740 in the manner P₁. At the same time, the spring 700 is configured to bias the needle mount 620 towards rotation about the axis 740 in the manner P₁. The syringe 200 is pre-filled with the drug and, thus, the openings 536, 564 in the cover 530 and carrier 550 can act to direct overflow drug to the hollow receptacle 720 via the opening 728 in the relief cap 710. The user can remove the relief cap (see also the relief cap 400) to expose the distal end 84 of the needle 80.

That said, depressing the clip 600 into the cover 520 in the manner D pivots the clip about the axis 740 relative to the needle mount 620 and carrier 550 in the manner P₁. This causes the proximal end 82 of the needle 80 to pass through the seal 66 and into the chamber 64 (FIG. 18). This automatically initiates the vitreous removal step through the distal end 84 of the needle 80.

At the same time, depressing the clip 600 in the manner D causes the guide members 616 to interact with the slider 650. In particular, the guide members 616 move along an arc centered on the axis 740 as the clip 600 pivots. The slider 650 is longitudinally movable along and relative to the arm 570. That said, as the clip 600 pivots in the manner P₁, the projections 666 slide within the respective guide members 616, causing the slider 650 to slide distally along the arm 570 and towards the axle 574. In doing so, the second projections 670 on the slider 650 move into alignment with the respective guide members 640 on the needle mount 620. The first projections 666 therefore cooperate with the guide members 640 to “self-align” the second projections 670 with the guide members 640.

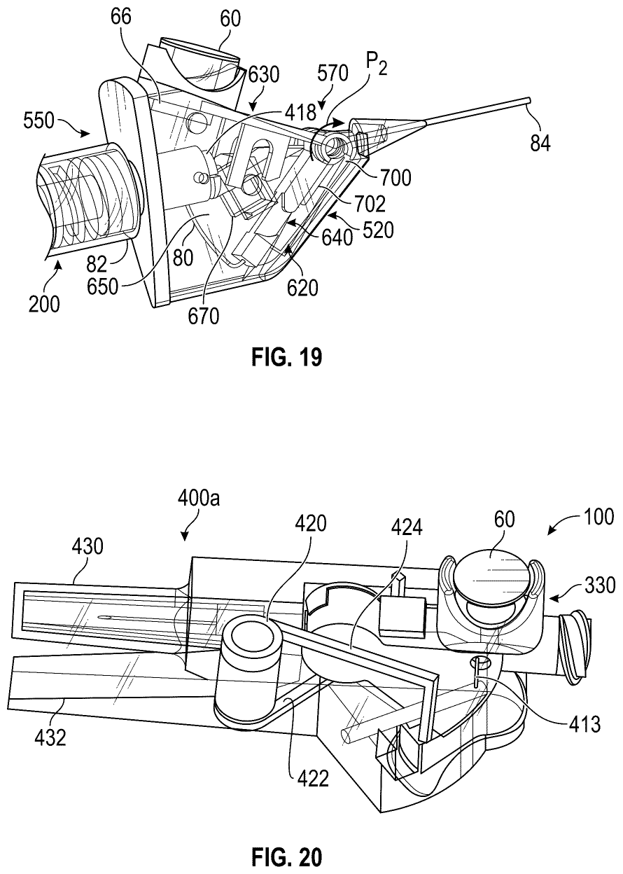

If the user then releases the clip 600, the arm 704 of the spring 700 causes the clip to pivot in the manner P₂ relative to the needle mount 620 and return to its initial position. A stop provided on the carrier 550 limits the return of the clip 600 under the bias of the spring 700. At the same time, since the spring 700 biases the needle mount 620 in the direction P₁, once the second projections 670 are aligned with the guide members 640 the needle mount 620 is capable of pivoting in the manner P₁ relative to the clip 600 and slider 650 as the slider slides proximally along the arm 570 and away from the axle 574. When this occurs, the second projections 670—under the bias of the arm 702—pass through the guide members 640 as the needle mount 620 pivots towards the cover 520. The needle 80 extends the length of the needle mount 620 and, thus, pivoting the needle mount in the manner P₁ causes the needle to bend about its radius of curvature (see FIG. 19).

More specifically, the proximal end 82 and the portion of the needle 80 within the passage 630 bends about the radius of curvature relative to the remainder of the needle extending through the passage 630 and the guide member 540 of the cover 520. The proximal end 82 now places the needle 80 in fluid communication with the drug in the syringe 200, which can then dispense a therapeutic to the eye.

In another example shown in FIG. 20, the relief cap 400a is configured as a spring-biased protective cap. To this end, the relief cap 400a includes a compression spring 420 having a pair of arms 422, 424 that bias a pair of arms 430, 432 of the cap away from one another. Consequently, the user urges the arms 430, 432 together to overcome the spring 420 bias and remove the relief cap 400a from the device. It will be appreciated that the relief cap 400a could likewise be used on the device 500.

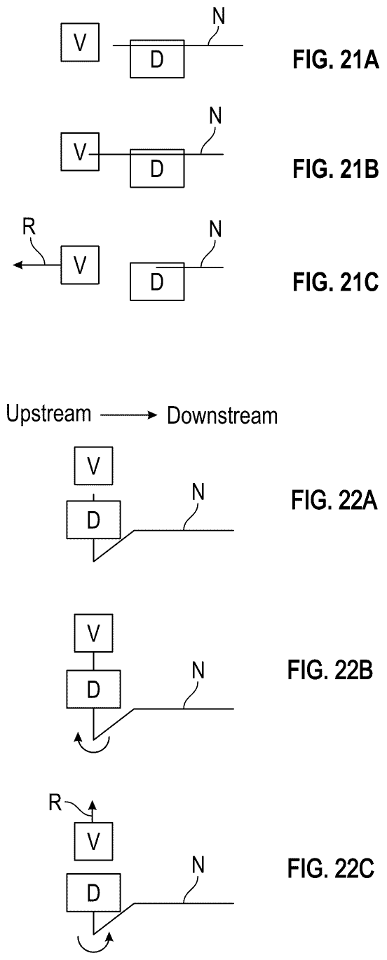

FIGS. 21A-21C illustrate in a broader sense the operation of the present invention. To this end, the vacuum sample assembly V is positioned on the upstream side or proximal of the drug delivery structure D. It will be appreciated that the vacuum sample assembly V can be the evacuated sample chamber 64 shown and described above or be formed as a hand-actuated vacuum having a piston slidable relative to the carrier to generate a vacuum.

In any case, the needle N initially extends through the drug delivery structure D to a location fluidly isolated from both the vacuum sample assembly V and the drug delivery structure D (FIG. 21A). Manipulation, e.g., axial movement, of the proximal end of the needle N in an upstream direction causes the proximal tip of the needle to enter the vacuum sample assembly V to automatically initiate vitreous sampling (FIG. 21B). Subsequent manipulation of the proximal end of the needle N in a downstream direction moves the proximal end into the drug delivery structure D to enable delivery of the therapeutic agent to the vitreous removal site (FIG. 21C). The vacuum sample assembly V can be readily removed (in the general manner R) from the device without disturbing the drug delivery structure D.

FIGS. 22A-22C illustrate in a broader sense alternative operation of the present invention. To this end, the vacuum sample assembly V is positioned on the upstream side or proximal of the drug delivery structure D. The needle N initially extends through the drug delivery structure D to a location fluidly isolated from both the vacuum sample assembly V and the drug delivery structure D (FIG. 22A). Bending of the proximal end of the needle N in a first manner (clockwise as shown) causes the proximal tip of the needle to enter the vacuum sample assembly V to automatically initiate vitreous sampling (FIG. 22B). Subsequent bending of the proximal end of the needle N in a second manner (counterclockwise as shown) moves the proximal end into the drug delivery structure D to enable delivery of the therapeutic agent to the vitreous removal site (FIG. 22C). The vacuum sample assembly V can be readily removed (in the general manner R) from the device without disturbing the drug delivery structure D.

The combined biological sampling and injection assemblies described herein are advantageous in that they provide for a small gauge, e.g., 30 G or smaller, instrument that can directly penetrate the sclera, does not require protective measures for the sclera, and can be distally operated in a cordless manner. Furthermore, the assemblies are disarmed until the cover is removed.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A vitrectomy device comprising:
a carrier having a pocket;
a cover connected to the carrier with at least one of the carrier and the cover having an axle defining an axis;
a trigger pivotably connected to the axle and having a passage;
a hollow needle for insertion into the vitreous humor of an eye and extending within the passage; and
a vacuum sample assembly received in the pocket and having a chamber under vacuum, wherein the trigger is pivotable about the axle from a first position in which the chamber is not fluidly connected to the needle to a second position in which the needle is bent into fluid communication with the chamber for automatically drawing a vitreous sample through the needle into the chamber under vacuum pressure.

2. The device recited in claim 1, wherein each of the carrier and the cover includes an axle aligned with one another for receiving a projection on the trigger to pivotably connect the trigger to the axles.

3. The device recited in claim 2, wherein the needle is spaced from and extends between the axles.

4. The device recited in claim 1, further comprising a syringe coupled to the carrier and having a drug disposed therein, wherein the trigger is pivotable to a third position in which the needle is bent into fluid communication with the syringe for delivering the drug to the eye.

5. The device recited in claim 4, wherein the trigger is pivoted in a first direction to reach the second position and a second direction opposite the first direction to reach the third position.

6. The device recited in claim 1, wherein the distal end of the needle comprises a sharpened tip configured for insertion into or through at least one of the sclera, the pars plana, the anterior segment or the vitreous body of the eye.

7. The device recited in claim 4, further comprising a relief cap having a needle for routing excess drug from the syringe into the relief cap.

8. The device a recited in claim 7, wherein the relief cap includes a pair of arms biased away from one another for securing the relief cap on the device.

9. A vitrectomy device comprising:
a clip having a pocket;
a vacuum sample assembly received in the pocket and having a chamber under vacuum;
a carrier having an axle for pivotably receiving the clip;
a needle mount pivotably mounted on the axle and including a passage;
a hollow needle for insertion into the vitreous humor of an eye and extending within the passage; and
a cam system configured to convert pivotal movement of clip into pivotal movement of the needle mount, the needle having a first condition fluidly isolated from the chamber when the clip has a first position relative to the needle mount and a second condition in which the needle is fluidly connected with the chamber in response to pivotal movement of the clip in a first direction for drawing a vitreous sample through the needle into the chamber under vacuum pressure.

10. The device recited in claim 9, wherein the needle has a third condition in response to pivotal movement of the clip in a second direction in which the needle is bent out of fluid communication with the chamber and into fluid communication with a therapeutic agent to be delivered to the eye.

11. The device recited in claim 10, wherein the cam system includes an arm and a slider positioned on the arm, the slider having projections engaging the clip and moving longitudinally in response to pivotal movement of the clip to place the needle in the third condition.

12. The device recited in claim 10, wherein the needle mount includes guide members for receiving second projections on the slider, the guide members being configured such that the second projections have a first interaction with the guide members when the clip pivots in the first direction and a second, different interaction with the guide members when the clip pivots in the second direction.

13. A vitrectomy device for an eye, comprising:
a carrier;
a hollow needle for insertion into the vitreous humor of the eye and extending from an upstream end to a downstream end and through the carrier;
a vitreous sample collection assembly coupled to the carrier at a first location; and
a drug container coupled to the carrier at a second location downstream of the vitreous sample collection assembly relative to the extension of the needle; and
the upstream end of the needle having a first condition passing through the drug container and spaced from the sample collection assembly, a second condition extending into the sample collection assembly for removing a vitreous sample from the eye, and a third condition spaced from the sample collection assembly and positioned within the drug container for delivering a therapeutic agent in the drug container to the eye.

14. The device recited in claim 13, further comprising a trigger pivotably connected to the carrier and receiving the needle such that pivotal movement of the trigger places the upstream end of the needle in the second condition and the third condition.

15. The device recited in claim 14, wherein pivotal movement of the trigger bends the needle to place the upstream end of the needle in the second condition and the third condition.

16. The device recited in claim 13, further comprising a clip pivotably connected to the carrier and receiving the sample collection assembly such that pivotal movement of the clip places the upstream end of the needle in the second condition and the third condition.

17. The device recited in claim 13, wherein the sample collection assembly includes a chamber under vacuum.

18. The device recited in claim 13, wherein the sample collection assembly includes a piston slidable relative to the carrier to generate a vacuum.

19. The device recited in claim 13, wherein the vacuum sample assembly is removable from the device without moving the drug container.
